(19) United States
(12) Patent Application Publication
(10) Pub. No.: US 2024/0352039 A1
(43) Pub. Date: Oct. 24, 2024

(54) NOVEL CRYSTALLINE POLYMORPH OF LURBINECTEDIN AND IMPROVED PROCESS FOR THE PREPARATION OF LURBINECTEDIN

(71) Applicant: NATCO PHARMA LIMITED, Hyderabad (IN)

(72) Inventors: Srinivasa Chary CHINTALAPATI, Hyderabad (IN); Thirupathi KOTTE, Hyderabad (IN); Srinivasan ABAYEE KALIYAPERUMAL, Hyderabad (IN); Shankar Reddy BUDIDETI, Hyderabad (IN); Pulla Reddy MUDDASANI, Hyderabad (IN); Venkaiah Chowdary NANNAPANENI, Hyderabad (IN)

(21) Appl. No.: 18/686,936

(22) PCT Filed: Aug. 30, 2022
(86) PCT No.: PCT/IN2022/050772

§ 371 Date: Feb. 27, 2024

(30) Foreign Application Priority Data
Aug. 31, 2021 (IN) 202141039393

(51) Int. Cl. C07D 515/22

(52) U.S. Cl. CPC C07D 515/22

(57) ABSTRACT

The present invention is related to novel crystalline polymorph. Form-N of Lurbinectedin and process for preparation of Lurbinectedin.

### FIELD OF THE INVENTION

The present invention relates to novel stable crystalline polymorph, Form-N of Lurbinectedin. The present invention also relates to an improved and industrially viable process for the preparation of Ecteinascidin derivative i.e., Lurbinectedin.

### BACKGROUND OF THE INVENTION

Lurbinectedin is an Ecteinascidin Derivative. Lurbinectedin is chemically known as (1′R,6R,6aR,7R,13S,14S,16R)-8,14-dihydroxy-6′,9-dimethoxy-4,10,23-trimethyl-19-oxo-2′,3′,4′,6,7,9′,12,13,14,16-decahydro-6aH-spiro[7,13-azano-6,16-(epithiopropanooxymethano)[1,3]dioxolo[7,8]isoquinolino[3,2-b][3] benzazocine-20,1′-pyrido[3,4-b]indol]-5-yl acetate and having the structure below

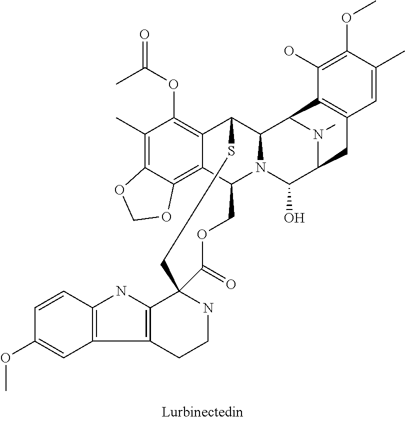 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

Lurbinectedin is approved under the brand name “ZEPZELCA” and it is indicated for the treatment of adult patients with metastatic small cell lung cancer (SCLC) with disease progression on or after platinum-based chemotherapy.

The U.S. Pat. No. 7,763,615 discloses synthesis and characterization of Lurbinectedin.

Angew Chem Int Ed Engl. 2019 Mar. 18; 58 (12): 3972-3975 has reported process for the preparation of Trabectedin and Lurbinectedin, wherein compound III was prepared from compound-IV by using 4-formyl-1-methylpyridiniumbenzenesulfonate, the reaction is not meeting large-scale requirement as it is giving low yield as well as inconsistent for completion of reaction. It also observed that the quality of 4-formyl-1-methylpyridinium benzenesulfonate is very important for the reaction success. Commercial availability of ultra-quality of 4-formyl-1-methylpyridiniumbenzenesulfonate is always risk.

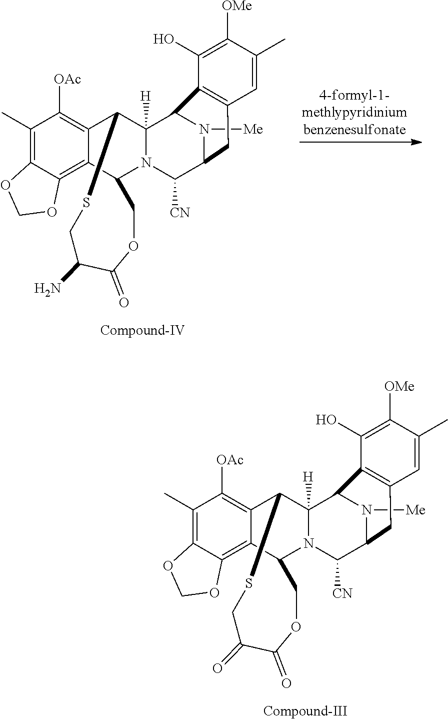

The patent application WO2021/099635 A1 described about amorphous form-A and Form-B. Compared to Form-A, Form-B is shown advantages of physical properties as per this patent application.

The inventors of the present invention have developed an alternative improved process for the preparation of Lurbinectedin. The present process is simple, cost effective and feasible in large scale production.

The inventors of the present invention also have developed novel and stable polymorph for Lurbinectedin and commercially viable process for the preparation of Lurbinectedin.

### OBJECT OF THE INVENTION

One object of the present invention is to provide a process for the preparation of Lurbinectedin, which is simple, economical, and suitable for industrial scale up.

Another objective of the present invention is to provide a novel and stable crystalline form-N of Lurbinectedin.

### SUMMARY OF INVENTION

Main aspect of the present invention relates to crystalline polymorph, Form-N of Lurbinectedin.

Another aspect of the present invention relates to crystalline polymorph, Form-N of Lurbinectedin characterized by its powder X-ray diffraction (PXRD) Pattern having peaks at about 4.8±0.2, 9.0±0.2, 9.5±0.2 and 11.8±0.2 2θ.

The process for the preparation of Lurbinectedin as per the present invention is depicted in the below scheme.

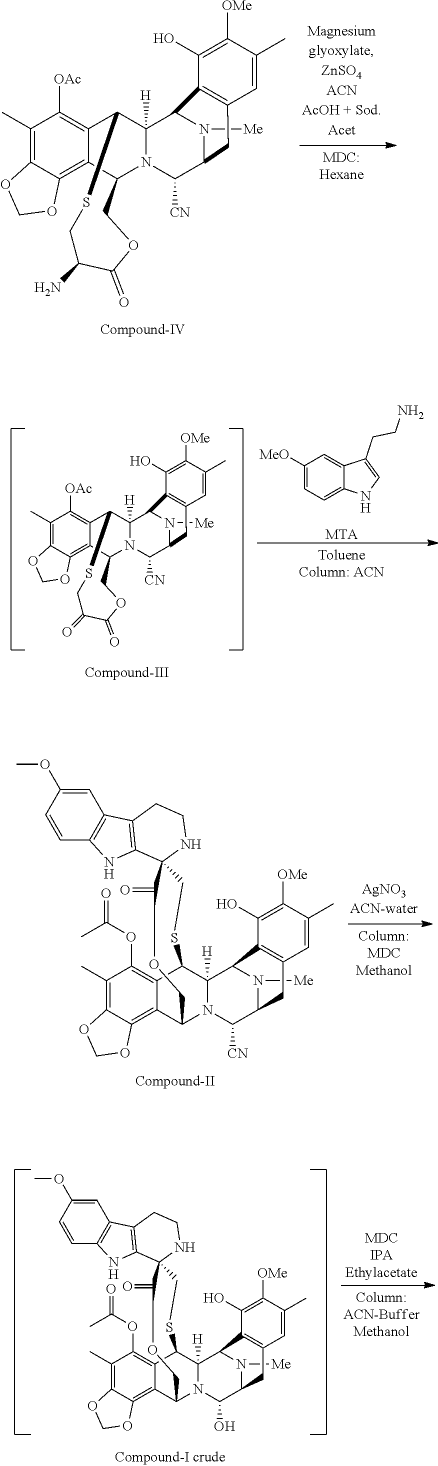

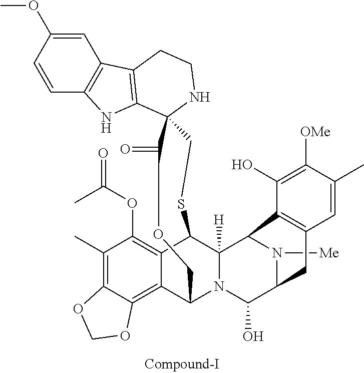

Yet another aspect of the present invention is related to process for the preparation of crystalline polymorph, Form-N of Lurbinectedin comprising the steps of:
a) dissolving Lurbinectedin in organic solvent or mixture thereof,
b) distil or co distil the organic solvent with or without vacuum to obtain suspension,
c) cool the suspension, filter and wash with organic solvent or mixture thereof to obtain crystalline polymorph, Form-N of Lurbinectedin.

### BRIEF DESCRIPTION OF THE DRAWINGS

FIG. 1: XRPD diffractogram of novel crystalline polymorph, Form-N of Lurbinectedin.

FIG. 2: DSC thermogram of novel crystalline polymorph, Form-N of Lurbinectedin.

FIG. 3: Infrared spectrum of novel crystalline polymorph, Form-N of Lurbinectedin.

FIG. 4: Thermogravimetric analysis of novel crystalline polymorph, Form-N of Lurbinectedin.

### DETAILED DESCRIPTION OF THE INVENTION

Main embodiment of the present invention relates to crystalline polymorph, Form-N of Lurbinectedin.

Another embodiment of the present invention relates to crystalline polymorph, Form-N of Lurbinectedin characterized by its powder X-ray diffraction (PXRD) Pattern having peaks at about 4.8±0.2, 9.0±0.2, 9.5±0.2 and 11.8±0.2 2θ.

Yet another embodiment of the present invention related to process for the preparation of crystalline polymorph, Form-N of Lurbinectedin comprising the steps of:
a) dissolving Lurbinectedin in organic solvent or mixture thereof,
b) distil or co distil the organic solvent with or without vacuum to obtain suspension,
c) cool the suspension, filter and wash with organic solvent or mixture thereof to obtain crystalline polymorph, Form-N of Lurbinectedin.

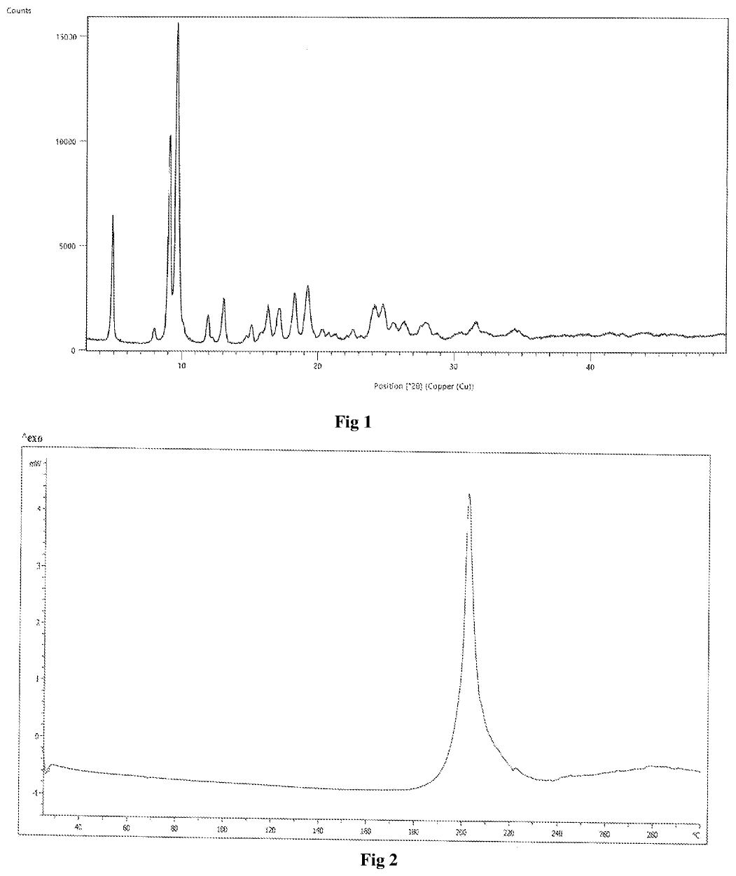

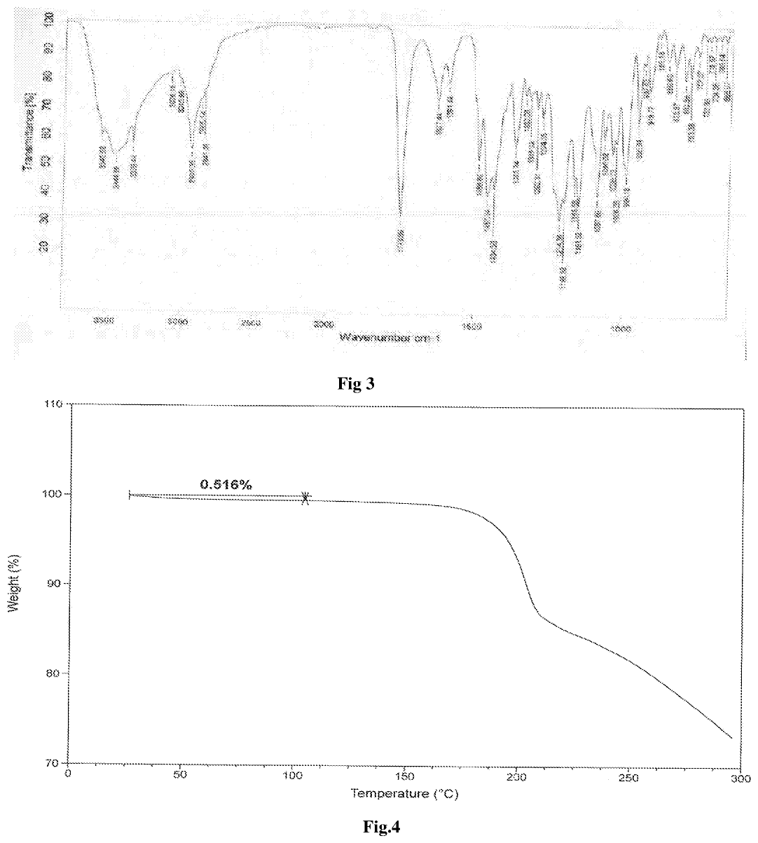

In most preferred embodiment, the present invention relates to crystalline polymorph, Form-N of Lurbinectedin that exhibits an PXRD pattern as shown in FIG. 1.

In addition, Form-N of Lurbinectedin can be characterized by DSC as shown in FIG. 2.

In another embodiment, Form-N of Lurbinectedin is anhydrous in nature.

In another embodiment, Form-N of Lurbinectedin has a water content less than 1% w/w.

As per the present invention preparation of Compound-IV was dissolved in organic solvent preferably acetonitrile and a freshly prepared buffer solution by using NaOAc and AcOH. In addition above, anhydrous zinc sulfate and metal glyoxylate or mixture of glyoxylate or thereof preferably Magnesium glyoxylate. The reaction mass was stirred 25-30° C., the reaction completion was monitored and diluted with organic solvent preferably Dichloromethane; organic layer was washed with water. The organic layer was concentrated, and the crude compound was isolated by from hexane and in-situ intermediate Compound-III was prepared.

As per the present invention preparation Compound-III was suspended in organic solvent preferably toluene to that 5-methoxy tryptamine and organic acid preferably acetic acid was added in the reaction mass and reaction mass was maintained at room temperature followed by 40-45° C. and monitored the reaction by HPLC. Insoluble was removed by filtration at 40-45° C. filtrate was washed with DM water. Crude was purified by chromatography to afford Compound-II.

A solution of Compound-II was dissolved in aqueous acetonitrile and Silver nitrate was added portion wise at 20-23° C., further maintained the reaction. The progress of the reaction was monitored by HPLC. The reaction mass was extracted with Dichloromethane afforded crude Compound-I.

Later Lurbinectedin is purified by chromatography, pure fraction further diluted with Dichloromethane and concentrated under reduced pressure up to 90% and to the syrupy solution Isopropyl alcohol is added and stirred for 10 minutes and co distilled twice with Isopropyl alcohol. The product was suspended in Isopropyl alcohol and stirred at RT for an hour; again concentrated the mass and co distil with ethyl acetate thrice. Finally to the residue added ethyl acetate and stirred at room temperature for 1 h & stirred for 2 h 0-5° C., filtered the solid and washed with precooled ethyl acetate dried the compound at 30-35° C. for 12 h to afford Compound-1. Obtained compound exhibits with novel crystalline polymorph, Form-N of Lurbinectedin with more than 99% purity by HPLC.

The novel crystalline polymorph, Form-N of Lurbinectedin is used in pharmaceutical composition preparation such as solutions, lyophilized compositions, etc., with suitable excipients for intravenous administration.

Inventors of the present application have come across a novel crystalline form of Lurbinectedin. Which is consistently reproducible, does not have the tendency to convert to other forms, and found to be more stable.

### Advantages of the Present Invention

1. The process of the present invention is feasible to produce on commercial scale of Lurbinectedin without any apprehension.
2. Novel crystalline polymorph, Form-N of Lurbinectedin is stable.
3. Novel crystalline polymorph, Form-N is stable at ambient temperature and at elevated temperatures.
4. The novel crystalline polymorph, Form-N of Lurbinectedin is substantially anhydrous and is stable at ambient storage conditions.

### PXRD Method of Analysis:

PXRD analysis of the crystalline form-N Lurbinectedin were carried out using Panlytical Expert Pro DY3248 X-ray powder diffractometer using Cu-Ka radiation of 10 wavelength 1.5406 A° and at continuous scan speed of 0.03°/min.

### DSC Method of Analysis:

Differential scanning calorimetric (DSC) analysis was performed with TA/2500 Discovery. Samples of about 2 to 3 milligrams held in a Tzero Aluminum Hermetic closed pan were analyzed at a heating rate of 10° C. per minute.

The Present invention is further illustrated in detail with reference to following examples. It is desired that the examples be considered in all respect as illustrative and are not intended to limit the scope of the invention in any way.

### Experimental Procedure

### Preparation of Lurbinectedin

### Example 1: Process for the Preparation of Compound-III

Compound-IV (6.5 g) is dissolved in acetonitrile at 25-30° C., treated with magnesium glyoxylate (10.6 g) in the presence of sodium acetate buffer solution & zinc sulphate. After completion of the reaction, it is quenched into a mixture of dichloromethane-DM water. Separated the layers, the aqueous layer is extracted with dichloromethane. The combined organic layer is washed with DM water, dried over anhydrous sodium sulphate, and concentrated under a vacuum. The foamy solid is co-distilled with hexanes to afford the compound.

### Example 2: Process for the Preparation of Compound-II

Compound-III (5.8 g) is reacted with 5-methoxytryptamine (2.2 g) in presence of acetic acid (0.84 g) at 25-30° C. in distilled toluene. The reaction mass is initially stirred at 25-30° C. for about 5 h and followed by at 40-45° C. for about 16 h. The reaction is monitored by HPLC. Upon completion of the reaction, insoluble mass is filtered. The filtrate is washed with DM water and the aqueous layer is extracted with distilled toluene. The combined organic layer is washed with DM water and concentrated under a vacuum to yield a crude compound-II. The crude product is further purified by Flash chromatography to yield pure compound-II. Weight: 4.4 g, Yield 60%

### Example 3: Process for the Preparation of Novel Crystalline Polymorph, Form-N of Lurbinectedin

Compound-II (4.2 g) is reacted with silver nitrate (13.48 g) in presence of aq. acetonitrile at 20-23° C. and the reaction is monitored by HPLC analysis. After completion of the reaction, the reaction mass is quenched into the mixture of dichloromethane-15% aq. sodium chloride solution-8% aq. sodium bicarbonate solution at 5-10° C. and insoluble mass are filtered through hyflo. The organic layer is separated, and aq. layer back extracted with dichloromethane. The combined organic layer is washed with DM water, dried over anhydrous sodium sulphate, and filtered. The filtrate is concentrated on rota vapor under a vacuum at below 25° C. to yield crude Lurbinectedin compound. The crude product is purified by flash chromatography and pure fractions are extracted with dichloromethane. The organic layer is concentrated on rota vapour and co-distilled with distilled isopropyl alcohol followed by ethyl acetate. The concentrated mass is treated with distilled ethyl acetate at 25-30° C., cooled to 0-5° C., filtered and dried at variable temperature to afford crystalline Form-N of Lurbinectedin.
Yield: 72%
Purity: 99.8%
Water content: 0.18% w/w

We claim:

1. Form-N of Lurbinectedin, characterized by its powder X-ray diffraction (PXRD) Pattern having peaks at about 4.8±0.2, 9.0±0.2, 9.5±0.2 and 11.8±0.2 2θ.

2. The form as claimed in claim 1, that exhibits an X-ray powder diffraction pattern substantially the same as of the X-ray powder diffraction patterns shown in FIG. 1.

3. The form as claimed in claim 1, that exhibits DSC as shown in FIG. 2.

4. The form as claimed in claim 1, is anhydrous in nature.

5. The form as claimed in claim 1, has a water content less than 1% w/w.

6. A process for the preparation of crystalline polymorph, Form-N of Lurbinectedin comprising the steps of:
a) dissolving Lurbinectedin in organic solvent or mixture thereof,
b) distil or co-distil the organic solvent with or without vacuum to obtain suspension,
c) cool the suspension, filter and wash with organic solvent or mixture thereof to obtain crystalline polymorph, Form-N of Lurbinectedin.

7. The process as claimed in claim 6, wherein the solvent is dichloromethane.

8. The process as claimed in claim 6, wherein the solvent is used as isopropyl alcohol and ethyl acetate.
